(19) United States
(12) Patent Application Publication
(10) Pub. No.: US 2017/0112393 A1
(43) Pub. Date: Apr. 27, 2017

(54) MEASUREMENT APPARATUS AND MEASUREMENT METHOD

(71) Applicant: KYOCERA Corporation, Kyoto (JP)

(72) Inventors: Tomoyuki TOUGASAKI, Yokohama-shi, Kanagawa (JP); Takeshi HIGUCHI, Yokohama-shi, Kanagawa (JP); Takaaki WADA, Yokohama-shi, Kanagawa (JP); Masato FUJISHIRO, Yokohama-shi, Kanagawa (JP)

(73) Assignee: KYOCERA Corporation, Kyoto (JP)

(21) Appl. No.: 15/316,934

(22) PCT Filed: Jun. 16, 2015
(86) PCT No.: PCT/JP2015/002999

§ 371 Date: Dec. 7, 2016

(30) Foreign Application Priority Data
Jun. 17, 2014 (JP) 2014-124413

(51) Int. Cl. A61B 5/026; A61B 5/00

(52) U.S. Cl. CPC A61B 5/0261; A61B 5/742; A61B 5/6826

(57) ABSTRACT

A measurement apparatus includes contact interfaces disposed in a housing so as to allow contact with test sites of a subject; biological sensors configured to acquire respective biological measurement outputs of the test sites contacting the contact interfaces; temperature detectors configured to detect respective temperatures of the contact interfaces; and a controller configured to measure biological information based on the temperatures obtained from the temperature detectors, and the biological measurement outputs obtained from the biological sensors.

### CROSS-REFERENCE TO RELATED APPLICATION

This application claims priority to and the benefit of Japanese Patent Application No. 2014-124413 filed Jun. 17, 2014, the entire contents of which are incorporated herein by reference.

### TECHNICAL FIELD

This disclosure relates to a measurement apparatus and a measurement method.

### BACKGROUND

An example of an existing measurement apparatus measures biological information by acquiring biological output information from a test site, such as a fingertip of a subject.

### SUMMARY

A measurement apparatus according to this disclosure includes:

a plurality of contact interfaces disposed in a housing so as to allow contact with a plurality of test sites of a subject;

a plurality of biological sensors configured to acquire respective biological measurement outputs of the test sites contacting the contact interfaces;

a plurality of temperature detectors configured to detect respective temperatures of the contact interfaces; and

a controller configured to measure biological information based on the temperatures obtained from the temperature detectors, and the biological measurement outputs obtained from the biological sensors.

While the solution to the problem in this disclosure has been described in terms of apparatuses, this disclosure may also be implemented as methods substantially corresponding to these apparatuses, and such methods are to be understood as included in the scope of this disclosure.

For example, a measurement method comprises:

acquiring a plurality of biological measurement outputs with a plurality of biological sensors from a plurality of test sites contacting a plurality of contact interfaces;

detecting temperatures of the plurality of the contact interfaces; and

measuring biological information based on the detected temperatures and on the acquired biological measurement outputs.

### BRIEF DESCRIPTION OF THE DRAWINGS

In the accompanying drawings:

FIG. 1 is an external perspective view schematically illustrating the structure of a measurement apparatus according to one of the embodiments of this disclosure;

FIG. 2 illustrates a subject holding the measurement apparatus of FIG. 1;

FIG. 3 is a functional block diagram schematically illustrating the structure of the measurement apparatus in FIG. 1;

FIG. 4 is a flowchart illustrating an example of processing executed by the controller in FIG. 1; and

FIGS. 5A and 5B illustrate examples of arrangement of the contact interfaces in the measurement apparatus.

### DETAILED DESCRIPTION

For example, a known blood flow measurement apparatus that measures blood flow as the biological information irradiates a fingertip with laser light and measures the blood flow based on scattered light from the blood flow in a capillary at the fingertip.

The measurement result of biological information, however, tends to change based on the pressure on the measurement apparatus from the test site. When the error in the measurement result of the biological information exceeds the allowable range, the subject needs to measure the biological information again, which the subject may find troublesome.

It would therefore be helpful to provide an improved measurement apparatus and measurement method.

The following describes one of the disclosed embodiments in detail with reference to the drawings.

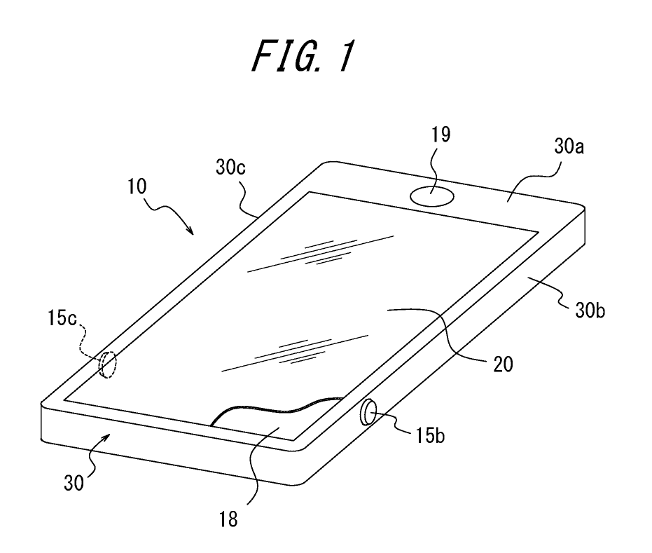

FIG. 1 is an external perspective view schematically illustrating the structure of a measurement apparatus according to one of the embodiments of this disclosure. This measurement apparatus 10 may be a measurement apparatus exclusively for measuring a subject's biological information, or an electronic device, such as a mobile phone, may be used as the measurement apparatus 10 according to this embodiment. The measurement apparatus 10 is not limited to a mobile phone and may be implemented in any type of electronic device, such as a portable music player, a laptop computer, a wristwatch, a tablet, a game device, or the like.

The measurement apparatus 10 according to this embodiment includes a housing 30 having an approximately rectangular external shape. In the housing 30, an input interface 19 and a panel 20 are provided at the front face 30a, and as illustrated by the partial cutout of the panel 20 in FIG. 1, a display 18 is held below the panel 20.

The panel 20 is configured using a touch panel that detects contact, a cover panel that protects the display 18, or the like and is, for example, made from glass or a synthetic resin such as acrylic or the like. The panel 20 is, for example, rectangular. The panel 20 may be a flat plate or may be a curved panel, the front face 30a of which is smoothly inclined. When the panel 20 is a touch panel, the panel 20 detects contact by the subject's finger, a pen, a stylus pen, or the like. Any detection system may be used in the touch panel, such as a capacitive system, a resistive film system, a surface acoustic wave system (or an ultrasonic wave system), an infrared system, an electromagnetic induction system, a load detection system, or the like. In the present embodiment, for the sake of explanation, the panel 20 is assumed to be a touch panel.

The measurement apparatus 10 according to this embodiment includes a contact interface 15b on a side face 30b, which is one of the long sides of the housing 30. The contact interface 15b is a portion of the below-described first measurement unit. The measurement apparatus 10 also includes a contact interface 15c on a side face 30c, which is the other long side of the housing 30, at a position symmetrical to the contact interface 15b when the measurement apparatus 10 is viewed from the front face 30a. The contact interface 15c is a portion of the second measurement unit. The contact interfaces 15b and 15c are portions that contact the test site, such as a finger, in order for the subject to measure biological information.

The input interface 19 receives operation input from the subject and may be configured, for example, using operation buttons (operation keys). The panel 20 can also receive operation input from the subject by detecting contact by the subject on a soft key or the like displayed on the display 18.

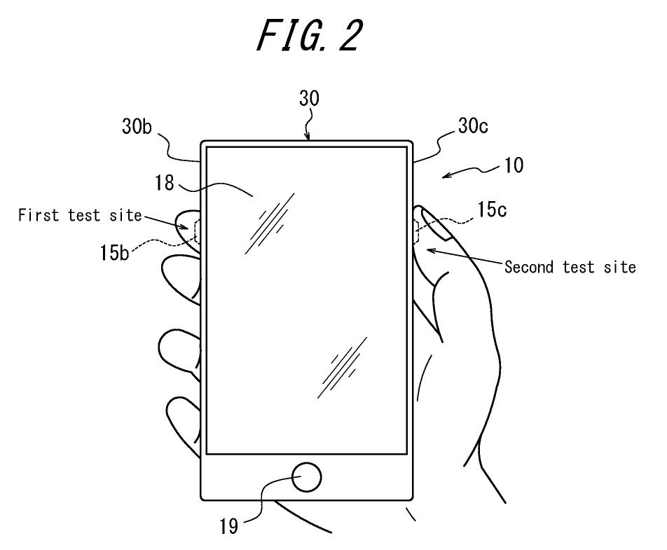

The measurement apparatus 10 measures biological information while being held by the subject. FIG. 2 illustrates the subject holding the measurement apparatus 10 of FIG. 1 in the right hand. In this case, for example as illustrated in FIG. 2, the pad of the right index finger contacts the contact interface 15b of the side face 30b, and the pad of the right thumb contacts the contact interface 15c of the side face 30c. The measurement apparatus 10 measures biological information while two fingers are being pressed against different contact interfaces 15b and 15c, as in FIG. 2. The biological information may be any biological information that can be measured using a biological sensor provided in the measurement apparatus 10. In this embodiment, as one example, the measurement apparatus 10 is described as measuring the subject's amount of blood flow, which is information related to blood flow.

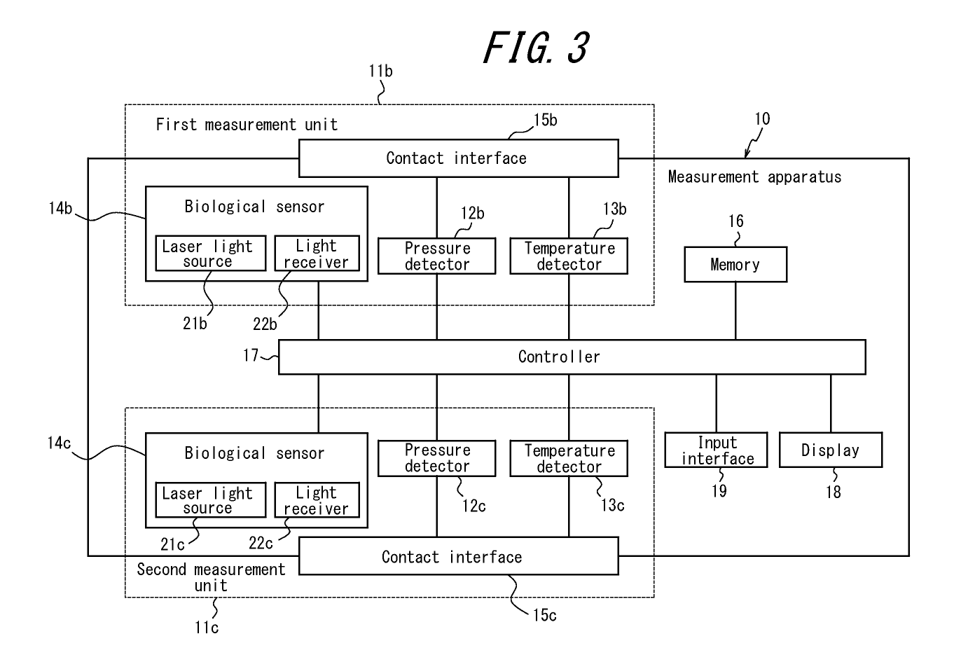

FIG. 3 is a functional block diagram schematically illustrating the structure of the measurement apparatus 10 in FIG. 1. As illustrated in FIG. 3, the measurement apparatus 10 includes a first measurement unit 11b, a second measurement unit 11c, a memory 16, a controller 17, the display 18, and the input interface 19. The first measurement unit 11b includes a pressure detector 12b, a temperature detector 13b, a biological sensor 14b, and the contact interface 15b. The second measurement unit 11c includes a pressure detector 12c, a temperature detector 13c, a biological sensor 14c, and the contact interface 15c. The pressure detectors 12b and 12c, the temperature detectors 13b and 13c, the biological sensors 14b and 14c, and the contact interfaces 15b and 15c respectively have the same functions. The biological sensor 14b includes a laser light source 21b and a light receiver 22b, and the biological sensor 14c includes a laser light source 21c and a light receiver 22c. The biological sensors 14b and 14c may, for example, be biological sensors of the same type. The contact interface 15b of the first measurement unit 11b is disposed on the side face 30b of the housing 30, and the contact interface 15c of the second measurement unit 11c is disposed on the side face 30c of the housing 30. Hereinafter, when not distinguishing whether each functional unit is included in the first measurement unit 11b or the second measurement unit 11c, the functional units are simply referred to as the pressure detector 12, temperature detector 13, biological sensor 14, contact interface 15, laser light source 21, and light receiver 22.

Each pressure detector 12 detects contact pressure, by the test site, acting on the corresponding contact interface 15. The pressure detector 12 may, for example, be configured by a piezoelectric element. The pressure detector 12 is connected to the controller 17 and transmits the detected contact pressure to the controller 17 as a pressure signal. Accordingly, when the test site is in contact with the contact interface 15, the pressure detector 12 detects the contact pressure, from the test site, acting on the contact interface 15 and transmits the detected contact pressure to the controller 17 as a pressure signal. When the subject holds the measurement apparatus 10 as illustrated in FIG. 2, the pressure detector 12b of the first measurement unit 11b detects the contact pressure acting on the contact interface 15b from the right index finger, and the pressure detector 12c of the second measurement unit 11c detects the contact pressure acting on the contact interface 15c from the right thumb.

Each temperature detector 13 detects the temperature of the corresponding contact interface 15. The temperature detector 13 may be configured by a known temperature sensor, such as a thermocouple, a thermistor, bimetal, or the like. The temperature detector 13 is connected to the controller 17 and transmits the detected temperature to the controller 17 as a temperature signal. Accordingly, when the test site is in contact with the contact interface 15, the temperature detector 13 detects the temperature of the contact interface 15 based on contact by the test site and transmits the detected signal to the controller 17 as a temperature signal. When the subject holds the measurement apparatus 10 as illustrated in FIG. 2, the temperature detector 13b of the first measurement unit 11b detects the temperature of the contact interface 15b based on contact by the right index finger, and the temperature detector 13c of the second measurement unit 11c detects the temperature of the contact interface 15c based on contact by the right thumb.

The biological sensor 14 acquires biological measurement output from the test site in contact with the contact interface 15. When the subject holds the measurement apparatus 10 as illustrated in FIG. 2, the biological sensor 14b of the first measurement unit 11b acquires biological measurement output from the right index finger, and the biological sensor 14c of the second measurement unit 11c acquires biological measurement output from the right thumb.

The laser light source 21 emits laser light based on control by the controller 17. The laser light source 21 may, for example, be configured to irradiate the test site with laser light, as measurement light, that has a wavelength capable of detecting a predetermined component included in blood. An example of such a laser light source 21 is a Laser Diode (LD).

The light receiver 22 receives scattered measurement light from the test site as biological measurement output. The light receiver 22 may, for example, be configured using a photodiode (PD). The biological sensor 14 transmits a photoelectric conversion signal of the scattered light received by the light receiver 22 to the controller 17.

As described above, the contact interface 15 is a portion that contacts the test site, such as a finger, in order for the subject to measure biological information. The contact interface 15 may, for example, be configured by a plate-shaped member. The contact interface 15 may also be configured by a member that is transparent at least with respect to the measurement light and the scattered light from the test site that is in contact.

The memory 16 may be configured with a semiconductor memory, a magnetic memory, or the like. The memory 16 stores a variety of information, programs for causing the measurement apparatus 10 to operate, and the like and also functions as a working memory. The memory 16 may, for example, store the amount of blood flow measured by the measurement apparatus 10 as history.

The controller 17 is a processor that, starting with the functional blocks of the measurement apparatus 10, controls and manages the measurement apparatus 10 overall. The controller 17 is configured using a processor such as a Central Processing Unit (CPU) that executes a program prescribing control procedures. Such a program may, for example, be stored in the memory 16, in an external storage medium, or the like.

The controller 17 causes laser light to be irradiated onto first and second test sites from the laser light sources 21b and 21c. The light receivers 22b and 22c acquire biological measurement outputs by receiving scattered light from the first and second test sites. The controller 17 determines whether the acquisition of the biological measurement output by the biological sensor 14 is complete. The controller 17 may, for example, judge that acquisition of the biological measurement output is complete once a predetermined length of time elapses after the biological sensor 14 starts to acquire the biological measurement output. The controller 17 may also, for example, judge that acquisition of the biological measurement output is complete once the biological sensor 14 has acquired sufficient biological measurement output to measure the biological information.

Based on outputs (biological information outputs) from the pressure detectors 12b and 12c, the temperature detectors 13b and 13c, and the light receiver 22, the controller 17 measures biological information. For example, the controller 17 selects one of the biological sensors 14b and 14c based on the contact pressures obtained from the pressure detectors 12b and 12c and the temperatures obtained from the temperature detectors 13b and 13c.

The selection of a biological sensor 14 based on the contact pressure obtained from the pressure detectors 12b and 12c is now described. For example, when the contact pressures by the subject on the contact interfaces 15b and 15c differ, i.e. when the contact pressures obtained from the pressure detectors 12b and 12c differ, it is envisioned that the measurement accuracy of the biological information measured based on the biological measurement outputs will also differ. The controller 17 judges whether the contact pressure obtained from the pressure detectors 12b and 12c is within a range (allowable range) such that the error in the measurement result of the amount of blood flow falls within a predetermined range.

Between the contact pressures obtained from the pressure detectors 12b and 12c, when one is within the allowable range, and the other is outside of the allowable range, the controller 17 selects the biological sensor 14b or 14c that corresponds to the pressure detector 12b or 12c that detected the contact pressure within the allowable range. When both of the contact pressures obtained from the pressure detectors 12b and 12c are within the allowable range, the controller 17 determines the pressure detector 12b or 12c indicating the contact pressure with the smaller measurement error in the biological information and selects the biological sensor 14b or 14c that corresponds to the determined pressure detector 12b or 12c. When both of the contact pressures obtained from the pressure detectors 12b and 12c are outside of the allowable range, the controller 17 determines the pressure detector 12b or 12c indicating the contact pressure that is closer to the allowable range and selects the biological sensor 14b or 14c that corresponds to the determined pressure detector 12b or 12c. The controller 17 then measures the biological information based on the biological measurement output obtained from the selected biological sensor 14b or 14c.

In this way, the measurement apparatus 10 detects contact pressures from a plurality of test sites, and when any of the contact pressures is within an allowable range, the controller 17 measures biological information based on the biological sensor 14b or 14c that corresponds to the pressure detector 12b or 12c indicating the contact pressure within the allowable range. When all of the contact pressures are within the allowable range or when all are outside of the allowable range, the controller 17 measures the biological information based on the biological sensor 14b or 14c that corresponds to the pressure detector 12b or 12c indicating the contact pressure that minimizes the measurement error in the biological information. Therefore, the measurement apparatus 10 can more easily obtain a highly accurate measurement result as compared to when biological information is measured based on one biological sensor. Furthermore, since the measurement apparatus 10 is provided with a plurality of biological sensors (two in this embodiment), it is more likely that at least one of the contact pressures will be included in the allowable range as compared to when biological information is measured based on one biological sensor.

The controller 17 can also select the biological sensor 14 based on the temperatures obtained from the temperature detectors 13b and 13c. Depending on the temperature of the test site, it may become difficult to output a highly accurate measurement result. Therefore, in addition to the above-described contact pressure, the controller 17 may select the biological sensor 14b or 14c based on the temperature detected by the temperature detectors 13b and 13c.

The controller 17 selects one of the biological sensors 14b and 14c based both on the above-described contact pressures obtained from the pressure detectors 12b and 12c and the temperatures obtained from the temperature detectors 13b and 13c. By having the controller 17 measure the biological information based on the biological measurement output obtained from the biological sensor 14b or 14c selected in this way, more accurate biological information can be expected to be output from the measurement apparatus 10 as a measurement result.

A technique for the controller 17 to measure the amount of blood flow using the Doppler shift is now described. When measuring the amount of blood flow, the controller 17 causes laser light to be irradiated from the laser light source 21 onto body tissue (the test site) and receives scattered light that is scattered from the body tissue with the light receiver 22. The controller 17 then calculates the amount of blood flow based on output related to the scattered light that was received.

In the body tissue, scattered light that is scattered from moving blood cells undergoes a frequency shift (Doppler shift), due to the Doppler effect, relative to the speed of travel of the blood cells within the blood. The controller 17 detects the beat signal due to interference between scattered light from still tissue and the scattered light from moving blood cells. This beat signal represents strength as a function of time. The controller 17 then turns the beat signal into a power spectrum that represents power as a function of frequency. In this power spectrum of the beat signal, the Doppler shift frequency is proportional to the speed of blood cells, and the power corresponds to the amount of blood cells. The controller 17 calculates the amount of blood flow by multiplying the power spectrum of the beat signal by the frequency and integrating.

The controller 17 then displays the biological information measured based on the biological measurement output obtained from the selected biological sensor 14b or 14c on the display 18. The subject can learn the amount of blood flow by confirming the display on the display 18.

At this time, the controller 17 may display whether the biological information is based on biological measurement output obtained from the biological sensor 14b or 14c, i.e. whether the biological information is based on biological measurement output of the index finger (first test site) or thumb (second test site) in contact with the contact interfaces 15b and 15c. By confirming such a display, a subject who is continuously measuring biological information can find out the test site for which biological measurement output is more easily selected, i.e. which test site more easily yields highly accurate biological measurement output.

In addition to the biological information measured based on biological measurement output obtained from the selected biological sensor 14b or 14c, the controller 17 may also measure the biological information based on the biological measurement output obtained from the biological sensor 14b or 14c that was not selected. Along with the biological information measured based on biological measurement output obtained from the selected biological sensor 14b or 14c, the controller 17 may also display, on the display 18, the biological information measured based on the biological measurement output obtained from the biological sensor 14b or 14c that was not selected. In this case, the controller 17 may display the selected biological sensor 14b or 14c.

The display 18 is a display device configured by a well-known display such as a liquid crystal display, an organic EL display, an inorganic EL display, or the like. For example, under control by the controller 17, the display 18 displays the measured biological information.

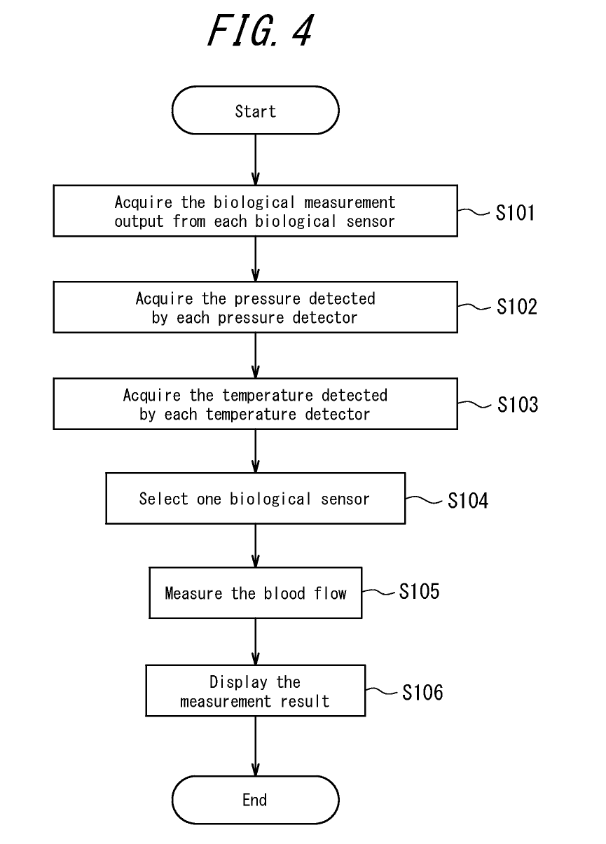

Next, with reference to the flowchart in FIG. 4, an example of processing executed by the controller 17 in FIG. 1 is described. The flowchart in FIG. 4 for example begins when the subject holds the measurement apparatus 10 so as to contact test sites to the contact interfaces 15 and performs an operation on the measurement apparatus 10 to place the measurement apparatus 10 in a state capable of measuring the amount of blood flow. In the flowchart in FIG. 4, as one example, the controller 17 is described as selecting one of the biological sensors 14b or 14c and displaying, on the display 18, the biological information measured based on the biological measurement output obtained from the selected biological sensor 14b or 14c.

The controller 17 causes the test sites to be irradiated with laser light from the laser light sources 21b and 21c and acquires biological measurement output from the biological sensors 14b and 14c (step S101).

The controller 17 acquires the contact pressure on the contact interfaces 15b and 15c as detected by the pressure detectors 12b and 12c (step S102).

The controller 17 acquires the temperature of the contact interfaces 15b and 15c as detected by the temperature detectors 13b and 13c (step S103).

Next, based on the acquired pressures and temperatures, the controller 17 selects one of the biological sensors 14b and 14c (step S104).

The controller 17 then measures the amount of blood flow as biological information based on the biological measurement output, between the biological measurement outputs acquired in step S101, that was obtained from the selected biological sensor 14b or 14c (step S105).

The controller 17 displays the amount of blood flow on the display 18 as the measurement result measured in step S105 (step S106).

In this way, the measurement apparatus 10 according to this embodiment acquires biological measurement outputs of a plurality of test sites from a plurality of biological sensors 14b and 14c. Based on the acquired pressures and temperatures, the controller 17 of the measurement apparatus 10 selects one biological sensor 14b or 14c that can measure the biological information with higher accuracy and measures the biological information based on the biological measurement output obtained from the selected biological sensor 14b or 14c. Therefore, with the measurement apparatus 10, the measurement accuracy of biological information improves. Even when the contact pressure on the contact interface 15b or 15c is outside of the allowable range, the measurement apparatus 10 can output highly accurate biological information without making the subject measure the biological information again.

This disclosure is not limited to the above embodiments, and a variety of modifications and changes are possible. For example, the functions and the like included in the various components and steps may be reordered in any logically consistent way. Furthermore, components or steps may be combined into one or divided.

For example, in the flowchart in FIG. 4, the controller 17 is described as acquiring the biological measurement output from the biological sensors 14b and 14c (step S101) and then selecting one of the biological sensors 14b and 14c based on the pressures and temperatures of the contact interfaces 15b and 15c (step 104), but the control by the controller 17 is not limited to this order. For example, the controller 17 may first select one of the biological sensors 14b and 14c based on the pressures and temperatures of the contact interfaces 15b and 15c and then acquire the biological measurement output of the selected biological sensor 14b or 14c. In this case, the controller 17 only causes laser light to be emitted from one of the laser light sources 21b and 21c. Hence, the power consumption can be suppressed as compared to when laser light is emitted from the laser light sources 21b and 21c.

In the above embodiment, the controller 17 has been described as selecting one of the biological sensors 14b and 14c based both on the contact pressures obtained from the pressure detectors 12b and 12c and the temperatures obtained from the temperature detectors 13b and 13c, but selection of a biological sensor is not limited to this method. For example, the measurement apparatus 10 may be configured so that the controller 17 selects one of the biological sensors 14b and 14c based only on the contact pressure detected by the pressure detectors 12b and 12c. In this case, the measurement apparatus 10 need not be provided with the temperature detectors 13 and can therefore have a simpler structure. The measurement apparatus 10 may be provided with another detector that detects information for selecting one biological sensor in order to improve the measurement accuracy of the biological information, and the controller 17 may select one biological sensor based on the information detected by this detector.

In the above embodiment, the measurement apparatus 10 has been described as being provided with first and second measurement units 11b and 11c, but the number of measurement units provided in the measurement apparatus 10 is not limited to two. The measurement apparatus 10 may include three or more measurement units 11. The controller 17 may, for example, select one biological sensor based on contact pressures on contact interfaces 15 in a plurality of measurement units 11 and measure the biological information based on the biological measurement output acquired by the selected biological sensor. As the number of measurement units 11 increases, the probability that the contact pressure on one of the contact interfaces will be included in the allowable range can be expected to increase.

In the above embodiment, the measurement apparatus 10 may notify the subject of information related to the strength of the contact pressures detected by the pressure detectors 12b and 12c. The information related to the contact pressures may, for example, be information related to whether the contact pressures are within the allowable range. The information related to the strength of the contact pressures may, for example, be information related to whether the contact pressures are stronger or weaker than the allowable range.

The measurement apparatus 10 can provide notification of information related to the strength of the contact pressures for example by a visual method using an image, characters, light emission, or the like; an auditory method using audio or the like; or a combination of these methods. The measurement apparatus 10 for example can provide notification of information related to the strength of the contact pressures via the display 18. The measurement apparatus 10 may, for example, include a separate notification interface that provides notification of information related to the strength of the contact pressures and may provide notification via the notification interface. Notification by the notification interface is not limited to a visual or auditory method. Any method recognizable by the user may be adopted. By the measurement apparatus 10 providing notification of information related to the strength of the contact pressures, the subject can find out what the contact pressures are when holding the measurement apparatus 10, making it easier to adjust the contact pressures to be within the allowable range.

In the above embodiment, the contact interfaces 15b and 15c have been described as being disposed respectively on the side faces 30b and 30c, but the arrangement of the contact interfaces 15b and 15c is not limited to this example. The contact interfaces 15b and 15c can, for example, be disposed separately at positions contacted by different fingers of the subject when the subject holds the housing 30 in one hand.

The contact interfaces 15b and 15c need not be disposed at positions that are determined assuming that the measurement apparatus 10 is held in one hand and may, for example, be disposed separately at positions contacted by different fingers of the subject when the subject holds the housing 30 in both hands.

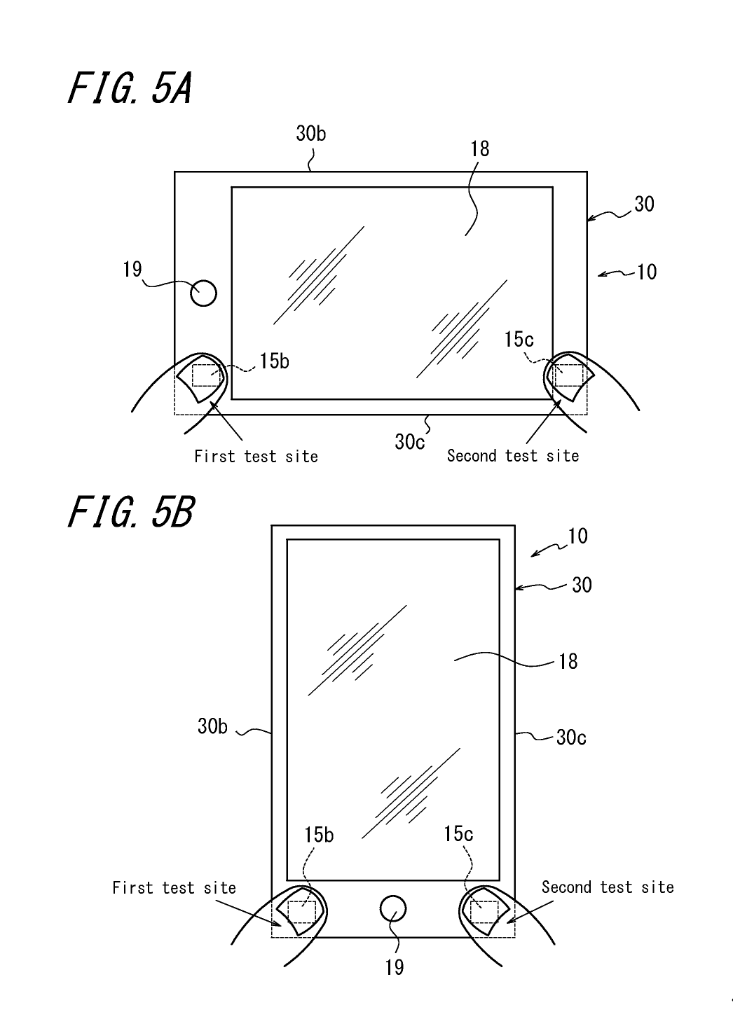

FIGS. 5A and 5B illustrate examples of arrangement of the contact interfaces 15 in the measurement apparatus 10. The contact interfaces 15b and 15c may, for example, be disposed on the front face 30a near one side face 30c at the top and bottom of the housing 30, as illustrated in FIG. 5A. In this case, the subject can measure biological information by contacting the pad of the left thumb as the first test site to the contact interface 15b and contacting the pad of the right thumb as the second test site to the contact interface 15c.

In another arrangement example, the contact interfaces 15b and 15c are disposed on the front face 30a respectively near the side faces 30b and 30c at the bottom left and right, as illustrated in FIG. 5B. In this case, the subject can measure biological information by contacting the pad of the left thumb as the first test site to the contact interface 15b and contacting the pad of the right thumb as the second test site to the contact interface 15c. Having the subject hold the measurement apparatus 10 in both hands in this way is useful when the measurement apparatus 10 is implemented in an electronic device that is difficult to hold in one hand, such as a tablet.

In the above embodiment, the controller 17 has been described as measuring the biological information based on the biological measurement output obtained from the selected biological sensor 14b or 14c, but the controller 17 is not limited to measuring the biological information with this method. The controller 17 may, for example, measure (calculate) the biological information using a predetermined algorithm that combines the biological measurement outputs from the biological sensors 14b and 14c by performing weighting determined based on the pressures obtained from the pressure detectors 12b and 12c and the temperatures obtained based on the temperature detectors 13b and 13c.

Furthermore, along with measuring a plurality of pieces of biological information based on the biological measurement outputs obtained from the biological sensors 14b and 14c, the controller 17 may also display, on the display 18, the result of comparing the measured pieces of biological information. The result of comparison is, for example, the difference in the amount of blood flow for each of the test sites.

Along with measuring a plurality of pieces of biological information based on the biological measurement outputs obtained from the biological sensors 14b and 14c, the controller 17 may also display the measured pieces of biological information on the display 18. At this time, among the measured pieces of biological information, the controller 17 may display biological information judged to have high measurement accuracy on the display 18. The biological information judged to have high measurement accuracy is biological information considered to be a more plausible representation of the user's biological information and for example is determined based on the contact pressure detected by the pressure detectors 12 and the temperatures detected by the temperature detectors 13. The controller 17 may display information related to whether each of the contact pressures detected by the pressure detectors 12b and 12c associated with the biological sensors 14b and 14c that measured the pieces of biological information is within the allowable range.

When judging that only one of the contact interfaces 15b and 15c among the contact interfaces 15 is being contacted based on the pressure obtained from the pressure detector 12b or 12c or the temperature obtained from the temperature detector 13b or 13c, the controller 17 may measure the biological information based on the biological measurement output from the biological sensor 14b or 14c corresponding to the contact interface 15b or 15c judged to be contacted.

The measurement apparatus 10 may further be provided with contact detection sensors near the contact interfaces 15b and 15c. The contact detection sensors are sensors that detect contact by the test site of the subject, such as a touch sensor, a pressor sensor, or a temperature sensor. When the controller 17 is configured to receive operation input from the subject by detecting contact from the subject on a soft key or the like displayed on the display 18, the controller 17 displays an image on the display 18 based on the positions of the contact interfaces 15b and 15c positioned near the contact detection sensors and on the output from the contact detection sensors. For example, suppose that the contact interfaces 15b and 15c are disposed at the positions indicated in FIG. 5B, and that the subject is pressing the contact interface 15c with the right thumb. At this time, upon the contact detection sensor disposed near the contact interface 15c detecting contact, the controller 17 judges that the subject is holding the measurement apparatus 10 with the right hand. The controller 17 then disposes soft key(s) displayed on the display 18 so as to be easily operable with the right hand, for example on the right side of the display 18. The right side of the display 18 refers, for example, to the right side relative to the center of the display 18 when the user is holding the measurement apparatus 10. Similarly, for example upon the contact detection sensor disposed near the contact interface 15b detecting contact, the controller 17 judges that the subject is holding the measurement apparatus 10 with the left hand and disposes soft key(s) displayed on the display 18 on the left side, for example, of the display 18. The left side of the display 18 refers, for example, to the left side relative to the center of the display 18 when the user is holding the measurement apparatus 10. As a result, when the subject measures biological information using the measurement apparatus 10 and then performs other operations using the measurement apparatus 10 while continuing to hold the measurement apparatus 10, the soft key(s) displayed on the display 18 are disposed near the finger performing the operations, thereby improving operability. The pressure detectors 12 or temperature detectors 13 may be used as the contact detection sensors.

In the above embodiment, the controller 17 of the measurement apparatus 10 has been described as measuring biological information, but measurement of biological information is not limited to being performed by the controller 17 of the measurement apparatus 10. For example, a measurement terminal provided with the measurement units 11 may transmit the biological measurement outputs acquired by the biological sensors 14 to a server that is connected to the measurement terminal by a network that is wired, wireless, or a combination of both. The server is provided with a server controller that executes similar control to that of the controller 17 in the above embodiment, and the server controller measures the biological information based on the biological measurement outputs. The measurement results are transmitted from the server to the measurement terminal and are displayed, for example, on a display of the measurement terminal.

1. (canceled)

2. (canceled)

3. A measurement apparatus comprising:
a plurality of contact interfaces disposed in a housing so as to allow contact with a plurality of test sites of a subject;
a plurality of biological sensors configured to acquire respective biological measurement outputs of the test sites contacting the contact interfaces;
a plurality of temperature detectors configured to detect respective temperatures of the contact interfaces; and
a controller configured to measure biological information based on the temperatures obtained from the temperature detectors, and the biological measurement outputs obtained from the biological sensors.

4. The measurement apparatus of claim 3, wherein the controller selects one of the biological sensors based on the temperatures obtained from the temperature detectors and measures the biological information based on the biological measurement output obtained from the selected biological sensor.

5. The measurement apparatus of claim 3, further comprising:
a display; wherein
the controller displays the measured biological information on the display.

6. The measurement apparatus of claim 3, further comprising:
a display; wherein
the controller measures a plurality of pieces of biological information based on the biological measurement outputs obtained from the biological sensors and displays the pieces of biological information on the display.

7. The measurement apparatus of claim 3, wherein the contact interfaces are disposed separately at positions contacted by a plurality of different fingers of the subject when the subject holds the housing in one hand.

8. The measurement apparatus of claim 3, wherein the contact interfaces are disposed separately at positions contacted by a plurality of fingers of different hands of the subject when the subject holds the housing in both hands.

9. The measurement apparatus of claim 3, further comprising:
a display; wherein
the controller measures a plurality of pieces of biological information based on the biological measurement outputs obtained from the biological sensors and displays a result of comparing the pieces of biological information on the display.

10. The measurement apparatus of claim 3, further comprising:
a display; and
a plurality of contact detection sensors disposed near the contact interfaces and configured to detect contact by the test sites of the subject; wherein
the controller displays an image on the display based on positions where the contact interfaces are disposed and on output from the contact detection sensors.

11. The measurement apparatus of claim 3, wherein the biological information includes information related to blood flow.

12. A measurement method comprising:
acquiring a plurality of biological measurement outputs with a plurality of biological sensors from a plurality of test sites contacting a plurality of contact interfaces;
detecting temperatures of the plurality of the contact interfaces; and
measuring biological information based on the detected temperatures and on the acquired biological measurement outputs.
